(19) United States
(12) Patent Application Publication
(10) Pub. No.: US 2012/0006348 A1
(43) Pub. Date: Jan. 12, 2012

(54) MULTI-COMPONENT ANTIPERSPIRANT AGENT COMPRISING TWO COMPONENTS CAPABLE OF COOPERATING, AND TWO-STEP PROCESS FOR TREATING HUMAN PERSPIRATION

(75) Inventors: Jean-Francois Grollier, Paris (FR); Henri Samain, Bievres (FR)

(73) Assignee: L'OREAL, Paris (FR)

(21) Appl. No.: 13/140,675

(22) PCT Filed: Dec. 21, 2009
(86) PCT No.: PCT/EP2009/067670

§ 371 Date: Sep. 13, 2011

Related U.S. Application Data
(60) Provisional application No. 61/142,245, filed on Jan. 2, 2009.

(30) Foreign Application Priority Data
Dec. 19, 2008 (FR) 0807300

(51) Int. Cl. A61K 8/18; A61Q 15/00; A61K 8/81; A61K 8/73; A61K 8/58; A61K 8/69

(52) U.S. Cl. 132/200; 424/65; 424/68

(57) ABSTRACT

The present invention relates to a multi-component antiperspirant agent comprising a first component formed from a cosmetic composition A and a second component formed from a cosmetic composition B different from the composition A, which are intended to be combined by application to the skin simultaneously, separately or sequentially over time, the said cosmetic composition A comprising one or more compounds that are capable of treating the skin, and a cosmetic composition B different from the cosmetic composition A, comprising one or more compounds that are capable of acting on the skin once it has been treated, the said combination being capable of imparting an antiperspirant effect. The present invention also relates to a process for treating perspiration using the multi-component antiperspirant agent, and also to the use of such an agent.

The present invention relates to a multi-component agent for treating human perspiration, comprising two components that are intended to be applied to the skin separately or sequentially over time, each of the said components being capable of cooperating together on contact with the skin to give an antiperspirant effect. The invention also relates to a two-step cosmetic process for treating human perspiration involving the use of the multi-compartment agent as defined above.

The armpits and also certain other parts of the body are generally the site of much discomfort that may arise directly or indirectly from perspiration. This discomfort often leads to unpleasant and disagreeable sensations that are mainly due to the presence of sweat resulting from perspiration, which may, in certain cases, make the skin and clothing wet, especially in the region of the armpits or on the back, thus leaving visible marks. Moreover, the presence of sweat may give rise to the production of body odour, which is generally unpleasant. Finally, during its evaporation, sweat may also leave salts and/or proteins at the surface of the skin, which may result in whitish marks on clothing. Such discomfort should be dealt with, including in the case of moderate perspiration.

In the cosmetic field, it is thus well known to use, in topical application, antiperspirant products containing substances that have the effect of limiting or even preventing the flow of sweat in order to overcome the problems mentioned above. These products are generally available in the form of roll-ons, sticks, aerosols or sprays.

Antiperspirant substances are generally formed from aluminium salts, such as aluminium chloride and aluminium hydroxyhalides, or complexes of aluminium and zirconium. These substances reduce the flow of sweat by forming a plug in the sweat duct.

However, the use of these substances at high concentrations for the purpose of obtaining good efficacy leads to formulation difficulties.

Furthermore, it has been found that the antiperspirant efficacy of these substances is limited when they are used alone. This means that these substances need to be applied regularly to the skin in order to obtain a satisfactory effective antiperspirant effect. However, it has been observed that repeated application of these substances may, in the case of certain users, lead to skin irritation.

Furthermore, these substances give antiperspirant effects that have a tendency to disappear, especially on washing and also in the case of substantial perspiration.

Finally, these antiperspirant substances may also leave marks during their application, which has the consequence of staining clothing.

As a variant, it has been proposed to develop a method that consists in using botulinum toxin to give an antiperspirant effect. However, the need to inject such a toxin at each use greatly limits the exploitation of this method.

To overcome all of the drawbacks mentioned above, it has been proposed to seek other effective active substances, which are well tolerated by the skin and easy to formulate, in order to replace all or some of the aluminium salts and/or aluminium and zirconium complexes.

Limiting the flow of sweat may be achieved by partially obstructing the sweat ducts by means of forming a plug in the sweat duct, but also by forming at the surface of the skin an adhesive or non-adhesive film that is resistant to sweat.

Thus, it has been proposed in international patent application WO 2001/054 658 to use anhydrous, non-adhesive antiperspirant compositions comprising at least one water-reactive cyanoacrylate monomer, an anhydrous medium, a polymerization inhibitor and an active substance chosen from a deodorant substance, an antiperspirant substance and a fragrancing substance, or a mixture of these substances. The cyanoacrylate monomers used in such compositions polymerize anionically directly on the surface of the skin in the presence of a nucleophilic agent, such as hydroxide ions (OH⁻) contained in water, to form a water-resistant polymer film. In other words, the cyanoacrylate monomers react with the sweat to form in situ, via anionic polymerization, a film on the skin that blocks the sweat ducts.

However, these occlusive film-forming polymers do not afford entirely satisfactory antiperspirant efficacy and still pose formulation problems.

Moreover, international patent application WO 2006/028 612 describes a process comprising a first step that consists in applying to the skin an adhesive silicone which then undergoes a heat treatment, and a second step that consists in applying an antiperspirant compound onto the surface of the adhesive. This method makes it possible to facilitate the application of the antiperspirant compound.

Patent application US 2007/0 053 959 describes a patch provided with a layer comprising an antiperspirant agent and/or a deodorant agent, on which is posed a protective layer. Thus, the method consists, in a first step, in applying the patch to the surface of the skin in order to deposit the antiperspirant agent and/or the deodorant agent, and, in a second step, in removing the protective layer.

However, it was observed that the films obtained with these cosmetic compositions are not fully tolerated by the skin, which usually leads to irritation problems in the case of certain users.

Finally, it has already been envisaged to use a process for treating human perspiration, which consists in applying to the skin, in a first stage, a first antiperspirant substance, and, in a second stage, a second antiperspirant substance, for the purpose of obtaining an improved and resistant antiperspirant effect.

However, it was observed that the application of a second antiperspirant substance generally has the drawback of impairing, reducing or even eliminating the antiperspirant effect imparted by the first substance, which especially has the consequence of reducing the staying power of the antiperspirant effect and also its efficacy.

Thus, there is a real need to use on the skin an agent intended for treating human perspiration, which does not have all the drawbacks described above, i.e. which gives a satisfactory antiperspirant effect, especially in terms of efficacy and resistance to sweat, and which is tolerated by the skin.

The Applicant has discovered, surprisingly, that by applying two different components to the skin, separately or sequentially over time, the first component being capable of treating the skin and the second component being capable of acting on the skin once it has been treated so as to impart an antiperspirant effect, it is possible to obtain a multi-component antiperspirant agent whose toxicological profile is suitable for the skin and which gives a satisfactory antiperspirant effect, especially in terms of efficacy and resistance to sweat.

In other words, the Applicant has found that the combination of a first component capable of preparing the skin so that a second component can act on the skin thus prepared to impart an antiperspirant effect, each of the said components having or not having an antiperspirant effect, makes it possible to lead to a satisfactory antiperspirant effect.

In other words, the first component is capable of treating the skin so that a second component can be active on the skin thus treated.

The first component is thus suited to preparing the skin, i.e. to chemically or physically modifying the skin, and the second component, which is different from the first component, is suited to acting on the skin thus prepared, i.e. it is active or its activity is reinforced on the skin once prepared so as to impart an antiperspirant effect. The second component is thus suited to profiting from the skin once it has been prepared.

The two components described above are thus selected so as to impart an antiperspirant effect.

In other words, it has been observed that the use on the skin of two different components that are capable of co-operating together on contact with the skin so as to impart an antiperspirant effect, the first component being capable of preparing the skin in order for the second component to be able to act on the skin once it has been prepared, makes it possible to impart a satisfactory and effective antiperspirant effect.

Thus, the multi-component agent according to the invention makes it possible to combine the application of two cosmetic compositions whose compounds are capable of co-operating together on contact with the skin so as to produce an action against perspiration, especially on the flow of sweat and also its effects, such as body odour or stains that may be produced on clothing, especially under the arms.

One subject of the present invention is thus especially a multi-component antiperspirant agent comprising a first component formed from a cosmetic composition A and a second component formed from a cosmetic composition B different from the composition A, which are intended to be combined by application to the skin separately or sequentially over time, the said cosmetic composition A comprising one or more compounds that are capable of treating the skin and the cosmetic composition B comprising one or more compounds that are capable of acting on the skin once it has been treated, the said combination being capable of imparting an antiperspirant effect.

The term “antiperspirant agent” means any substance which, by itself, has the effect of reducing or limiting the flow of sweat.

Another subject of the present invention also consists of a cosmetic process for treating human perspiration, which consists in combining by application to the skin separately or sequentially over time a cosmetic composition A and a cosmetic composition B different from the cosmetic composition A, the said cosmetic composition A comprising one or more compounds that are capable of treating the skin and the cosmetic composition B comprising one or more compounds that are capable of acting on the skin once it has been treated, the said combination being capable of imparting an antiperspirant effect.

Similarly, the present invention relates to the use of a multi-component antiperspirant agent as defined above for treating human perspiration.

The present invention also relates to a multi-compartment device or kit comprising a first compartment including a cosmetic composition A and a second compartment including a cosmetic composition B different from the cosmetic composition A, which are intended to be combined by application to the skin separately or sequentially over time, the said cosmetic composition A comprising one or more compounds that are capable of treating the skin and the said cosmetic composition B comprising one or more compounds that are capable of acting on the skin once it has been treated, the said combination being capable of imparting an antiperspirant effect.

Other subjects, characteristics, aspects and advantages of the invention will emerge even more clearly on reading the description and the examples that follow.

For the purposes of the present invention, the term “capable of treating the skin” means that the cosmetic composition A comprises one or more compounds that are capable of chemically or physically modifying the skin.

For the purposes of the present invention, the term “chemically modifying the skin” means that the cosmetic composition A comprises one or more compounds that are capable of reacting chemically with the skin so as to become grafted onto the skin or to create, especially, reactive sites, ionic groups, hydrophobic groups or catalytic sites on the skin.

For the purposes of the present invention, the term “physically modifying the skin” means that the cosmetic composition A comprises one or more compounds that are capable of modifying the physical properties of the skin in the absence of a chemical reaction with the skin.

The compound(s) of the cosmetic composition A are thus capable of chemically or physically modifying the skin such that the compound(s) of the cosmetic composition B can be active or such that their activity is reinforced on the pretreated skin so as to impart an antiperspirant effect.

According to one embodiment, the cosmetic composition A comprises one or more compounds that are capable of treating the skin by involving a chemical reaction.

For the purposes of the present invention, the expression “compounds that are capable of treating the skin by involving a chemical reaction” means that the cosmetic composition A comprises one or more compounds that are capable of inducing a chemical reaction with the skin, for example a chemical reaction with the skin proteins or with other compounds such as water or body fats in order thereafter to react with the skin.

The cosmetic composition A comprises one or more compounds chosen from compounds that are capable of grafting to the skin, compounds that are capable of opening one or more bonds of the skin, compounds that are capable of modifying the ionic nature of the skin, compounds that are capable of removing all or some of the molecules present in the skin, compounds that are capable of creating catalytic groups on the skin, or a mixture of these compounds.

According to a first aspect of this embodiment, the cosmetic composition A comprises one or more compounds that are capable of grafting to the skin.

In particular, the compound(s) of the cosmetic composition A include one or more functions capable of grafting to the skin, especially by reacting with the nucleophilic functions of the skin, such as the hydroxyl, mercaptan and amine functions of the skin.

In accordance with this embodiment, the compound(s) of the cosmetic composition A may include one or more functions capable of grafting to the skin, which may be chosen from the following functions:
epoxide,
aziridine,
vinyl and activated vinyl, in particular acrylonitrile and acrylic and methacrylic esters,
crotonic acid and crotonic esters, cinnamic acid and cinnamic esters, styrene and derivatives thereof, and butadiene,
vinyl ethers, vinyl ketone, maleic esters, vinyl sulfones and maleimides,
anhydride, acid chloride and carboxylic acid esters,
aldehydes,
acetals, hemiacetals,
aminals, hemiaminals,
ketones, α-hydroxy ketones, α-halo ketones,
lactones, thiolactones,
isocyanate,
thiocyanate,
imines,
imides, in particular succinimide and glutimide,
N-hydroxysuccinimide esters,
imidates,
thiosulfate,
oxazine and oxazoline,
oxazinium and oxazolinium,
C₁-C₃₀ alkyl halides or C₆-C₃₀ aryl or aralkyl halides of formula RX with X=I, Br or Cl,
halide of unsaturated carbon-based ring or heterocycle, especially chlorotriazine,
chloropyrimidine, chloroquinoxaline or chlorobenzotriazole,
sulfonyl halide: RSO₂Cl or F, R being a C₁-C₃₀ alkyl,
siloxane.

For the purposes of the present invention, the term “activated vinyl” means that the vinyl function has an asymmetrical electronic distribution and is thus more reactive.

In particular, the compound(s) of the cosmetic composition A may include one or more siloxane functions that may react, for example, with one or more hydroxyl or amine functions of the skin.

According to a second aspect of this embodiment, the cosmetic composition A comprises one or more compounds that are capable of opening one or more bonds of the skin, in particular the disulfide bonds or the peptide bonds of the keratin constituting the skin.

For the purposes of the present invention, the expression “opening the disulfide or peptide bonds of the keratin constituting the skin” means that the cosmetic composition A comprises one or more compounds that are capable of breaking the disulfide bonds (S—S) or the peptide bonds (carbon/nitrogen bonds between two amino acids) of keratin.

The compound(s) of the cosmetic composition A that are capable of opening one or more disulfide or peptide bonds of keratin may generate one or more reactive groups at the surface of the skin. In other words, such compounds are considered as being compounds that are capable of creating reactive groups at the surface of the skin.

Thus, when the cosmetic composition A comprising one or more compounds that are capable of opening one or more disulfide bonds of keratin is applied to the skin, one or more reactive groups are formed at the surface of the skin, such as thiol groups, thiosulfate groups, mixed disulfide groups and/or sulfonic acid groups. The term “mixed disulfide” means a compound in which the two groups containing two sulfur atoms of the disulfide are different and in particular do not produce the same electron-donating or electron-withdrawing effect. In this asymmetric manner, the mixed disulfide may be more reactive than a symmetrical disulfide.

Alternatively, when the cosmetic composition A comprises one or more compounds that are capable of opening one or more peptide bonds, for example by hydrolysing the peptide bonds of protein chains or by hydrolysing the pendent functions of proteins, and in particular the amide functions of glutamine and asparagine, one or more reactive groups are created by especially forming one or more amine and/or acid functions at the surface of the skin.

The compound(s) of the cosmetic composition A that are capable of opening one or more disulfide or peptide bonds of the skin may be chosen from reducing agents, acids, bases and oxidizing agents, and a mixture of these compounds.

The reducing agent(s) that may be used in the cosmetic composition A may be chosen from thiols, sulfinic acids, phosphines, sulfites and hydrides, or a mixture of these compounds.

As thiol reducing agents, it is especially possible to use thioglycolic acid, cysteine, homocysteine or thiolactic acid, and also the salts of these thiols.

Among the sulfinic acids, hydroxymethanesulfinic acid is preferably used. The cosmetically acceptable salts of the sulfinic acids are especially chosen from alkali metal (Na, K), alkaline-earth metal (Ca) or zinc sulfinates. Sodium hydroxymethanesulfinate is particularly preferred among the sulfinic acids that may be envisaged in the cosmetic composition A.

Among the sulfites, it is especially preferred to use alkali metal, alkaline-earth metal or ammonium salts, and also mixtures thereof. More particularly, mention may be made of sodium sulfite and sodium hydrogen sulfite.

Among the hydrides, it is preferred to use alkali metal or alkaline-earth metal salts such as sodium hydrides.

Among the phosphines, it is preferred to use tris(hydroxymethyl)phosphine, tris(hydroxypropyl)phosphine and tris(carboxyethyl)phosphine.

The acid(s) that may be used in the cosmetic composition A may be chosen from mineral or organic acids, for instance hydrochloric acid, orthophosphoric acid, sulfuric acid, carboxylic acids, for instance acetic acid, tartaric acid, citric acid or lactic acid, and sulfonic acids.

The base(s) that may be used in the cosmetic composition A may be chosen from aqueous ammonia, alkali metal carbonates, alkanolamines such as monoethanolamine, diethanolamine or triethanolamine, and also derivatives thereof, sodium hydroxide, potassium hydroxide and the compounds of formula (VI) below:

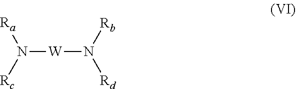

in which W is a propylene residue optionally substituted with a hydroxyl group or a C₁-C₄ alkyl radical; Rₐ, R_{b}, R_{c} and R_{d}, which may be identical or different, represent a hydrogen atom, a C₁-C₄ alkyl radical or a C₁-C₄ hydroxyalkyl radical.

The oxidizing agent(s) may be chosen from hydrogen peroxide, urea peroxide, alkali metal bromates, persalts such as perborates and persulfates, peracids, and oxidase enzymes (with the possible cofactors thereof), among which mention may be made of peroxidases, 2-electron oxidoreductases such as uricases, and 4-electron oxygenases, for instance laccases.

Preferably, the compound(s) of the cosmetic composition A that are capable of opening one or more disulfide bonds are chosen from sulfites, in particular sulfite salts, especially alkali metal or alkaline-earth metal sulfites such as sodium sulfite, and thioglycolic acid.

According to a third aspect of this embodiment, the cosmetic composition A comprises one or more compounds that are capable of modifying the ionic nature of the skin by creating ionic groups at the surface of the skin, such as anionic, cationic or zwitterionic groups, or by creating hydrophobic groups.

Preferably, the cosmetic composition A comprises one or more compounds that are capable of modifying the ionic nature of the skin by creating anionic or cationic groups at the surface of the skin.

In particular, the compound(s) of the cosmetic composition A may be chosen from cysteine salts that modify the ionic nature of the skin by creating anionic groups at the surface of the skin. Preferably, the cysteine salts are cysteine halides, such as cysteine hydrochloride.

The compound(s) of the cosmetic composition A that are capable of modifying the ionic nature of the skin by creating cationic groups may also be chosen from alkoxysilanes comprising a substituent including a primary amine function, which are chosen from the compounds of formula:

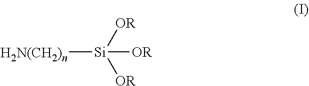

in which the radicals R, which may be identical or different, are chosen from C₁-C₆ alkyl radicals and n is an integer from 1 to 6 and preferably from 2 to 4.

An alkoxysilane that is particularly preferred according to this embodiment of the invention is γ-aminopropyltriethoxysilane. Such a compound is sold, for example, under the name Z-6011 Silane by the company Dow Corning.

The cosmetic composition A may also comprise one or more compounds chosen from cationic polymers.

Preferably, the cationic polymer(s) may be chosen from polymers that are formed from repeating units corresponding to formula (IX) below:

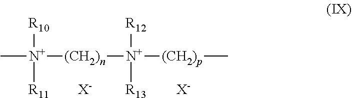

in which R₁₀, R₁₁, R₁₂ and R₁₃, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

(11) Poly(quaternary ammonium) polymers consisting of units of formula (X):

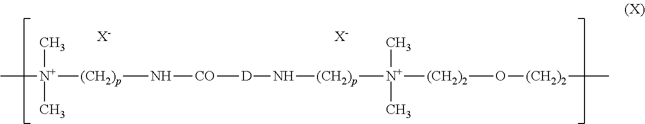

in which p denotes an integer ranging from 1 to 6 approximately, D can be zero or can represent a group —(CH₂)ᵣ—CO— in which r denotes a number equal to 4 or 7, and X⁻ is an anion.

Such polymers may be prepared according to the processes described in U.S. Pat. Nos. 4,157,388, 4,702,906 and 4,719,282. They are especially described in patent application EP-A-122 324.

More particularly, the preferred compound of formula (IX) is that in which R₁₃, R₁₄, R₁₅ and R₁₆ represent a methyl radical, A₁ represents the radical of formula —(CH₂)₃— and B₁ represents the radical of formula —(CH₂)₆— and X⁻ represents the chloride anion (referred to hereinbelow as Mexomer PO) and the compound of formula (IX) in which R₁₃ and R₁₄ represent an ethyl radical, R₁₅ and R₁₆ represent a methyl radical, A₁ and B₁ represent the radical of formula —(CH₂)₃— and X⁻ represents the bromide anion (referred to hereinbelow as Mexomer PAK).

Thus, the compound(s) of the cosmetic composition A that are capable of modifying the ionic nature of the skin are preferably chosen from cysteine salts, cationic polymers of formula (IX) and alkoxysilanes comprising a substituent including a primary amine function chosen from the compounds of formula (I) described above.

According to a fourth aspect of this embodiment, the cosmetic composition A comprises one or more compounds that are capable of removing all or some of the molecules present in the skin.

In this case, the compound(s) of the cosmetic composition A may especially react with water so as to dry out the skin. By way of example, the cosmetic composition A comprises one or more anhydrides, in particular organic or mineral anhydrides such as phosphate anhydride.

According to a fifth aspect of this embodiment, the cosmetic composition A comprises one or more compounds that are capable of generating one or more catalytic groups at the surface of the skin.

For example, the compound(s) of the cosmetic composition A correspond to compounds chosen from metal salts and enzymes that are grafted onto the skin.

Thus, the compound(s) of the cosmetic composition A are catalysts that become bound to the skin.

In particular, the compound(s) of the cosmetic composition A may be chosen from enzymes, in particular laccases, oxidases, dismutases, catalases, peroxidases and transition metal salts or other metal atoms.

Preferably, the compound(s) of the cosmetic composition A that are capable of creating catalytic groups at the surface of the skin are chosen from copper salts and manganese salts such as manganese chloride.

In accordance with the present invention, the cosmetic composition A may comprise one or more compounds that are capable of grafting onto the skin and one or more compounds that are capable of opening the disulfide bonds of keratin.

According to one variant, the cosmetic composition A may comprise one or more compounds that are capable of opening the disulfide bonds of keratin to generate one or more reactive groups and one or more compounds that are capable of generating catalytic groups on the skin.

Thus, the cosmetic composition A may comprise thioglycolic acid and one or more transition metal salts chosen from copper salts and manganese salts such as manganese chloride.

Thioglycolic acid makes it possible to create at the surface of the skin reactive groups such as thiols that form a bond with the catalysts bound to the skin, which have been formed from the transition metal salts. The bond obtained is resistant to water and to various washing operations.

According to another embodiment, the cosmetic composition A comprises one or more compounds that are capable of treating the skin not involving a chemical reaction with the skin.

For the purposes of the present invention, the expression “not involving a chemical reaction with the skin” means that the cosmetic composition A comprises one or more compounds that are capable of treating the skin without exerting a chemical action, i.e. the compounds do not induce any chemical reaction with the skin, either at the time of application of the cosmetic composition A, or thereafter. In other words, the cosmetic composition A comprises one or more compounds that are capable of modifying the physical properties of the skin in the absence of a chemical reaction therewith.

According to a first aspect of this embodiment, the cosmetic composition A comprises one or more compounds that are capable of increasing the retention on the skin of one or more compounds of the cosmetic composition B.

In accordance with this first aspect of this embodiment, the cosmetic composition A comprises one or more compounds chosen from oils, waxes, compounds that are capable of creating physical interactions with one or more compounds of the cosmetic composition B.

The term “oil” means a non-aqueous substance that is liquid at room temperature (22° C.±3° C.) and atmospheric pressure (760 mmHg).

The cosmetic composition A in accordance with the invention comprises one or more non-silicone oils that may be chosen in particular from hydrocarbon-based oils and fluoro oils, or mixtures thereof.

The term “hydrocarbon-based oil” means an oil mainly containing hydrogen and carbon atoms and possibly oxygen, nitrogen, sulfur or phosphorus atoms.

Hydrocarbon-based oils that may especially be mentioned include:
hydrocarbon-based oils of plant origin, such as triglycerides consisting of fatty acid esters of glycerol, the fatty acids of which may have varied chain lengths from C₄-C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810®, 812® and 818® by the company Dynamit Nobel,
synthetic ethers containing from 10 to 40 carbon atoms;
linear or branched hydrocarbon-based oils free of heteroatoms, of mineral or synthetic origin, such as liquid paraffin, polydecenes, hydrogenated polyisobutene such as Parleam, and squalane, and mixtures thereof;
liquid synthetic esters such as compounds of formula R₁COOR₂ in which R₁ represents a linear or branched radical containing from 1 to 40 carbon atoms and R₂ represents an in particular branched hydrocarbon-based chain containing from 1 to 40 carbon atoms, on condition that the sum of the carbon atoms of R₁ and R₂ is greater than or equal to 10, for instance purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;

and mixtures thereof.

The fluoro oils that may be used in the compositions in accordance with the invention are especially fluorinated polyethers.

Preferably, the oil used in the cosmetic composition A is chosen from a hydrocarbon-based oil of plant origin and liquid synthetic esters, preferably isopropyl myristate.

The wax(es) that may be used in the cosmetic composition A are chosen especially from carnauba wax, candelilla wax, esparto grass wax, paraffin wax, ozokerite, plant waxes, for instance olive wax, rice wax, hydrogenated jojoba wax or the absolute waxes of flowers such as the essential wax of blackcurrant blossom sold by the company Bertin (France), animal waxes, for instance beeswaxes, or modified beeswaxes (cerabellina); other waxes or waxy starting materials that may be used according to the invention are especially marine waxes such as the product sold by the company Sophim under the reference M82, and polyethylene waxes or polyolefin waxes in general.

According to a second aspect of this embodiment, the cosmetic composition A comprises one or more compounds that are capable of removing agents that are liable to harm the action of the compound(s) of the cosmetic composition B.

Thus, the cosmetic composition A comprises one or more compounds that are capable of removing elements that may be present in the skin pores.

According to a third aspect of this embodiment, the cosmetic composition A comprises one or more compounds that are capable of improving the distribution and/or penetration of one or more compounds of the cosmetic composition B on and/or in the skin.

In particular, the cosmetic composition A comprises one or more compounds that are capable of improving the penetration of one or more compounds of the cosmetic composition B into the skin or into the sweat ducts.

Thus, the cosmetic composition A comprises one or more compounds chosen from lubricants, agents that modify the surface tension of the skin, solubilizers, or agents capable of removing compounds that may be an obstacle to the action of the compound(s) of the cosmetic composition B.

The lubricants that may be used in the cosmetic composition A may be chosen from polyethylene glycols (e.g. PEG-14M and PEG-23M) and silicones such as dimethicone, dimethiconol, dimethicone copolyol, stearyl dimethicone, cetyl dimethicone copolyol or cyclomethicone.

The agents that modify the surface tension of the skin may be chosen from surfactants, in particular cationic, nonionic, anionic or amphoteric surfactants, in particular, those bearing cationic functions, such as genamin and betaines.

The solubilizers that may be used in the cosmetic composition A may be water, one or more organic solvents or a mixture of water and organic solvent.

Examples of organic solvents that may be mentioned include C₁-C₄ lower alkanols, such as ethanol and isopropanol; polyols and polyol ethers, for instance 2-butoxyethanol, propylene glycol, glycerol, propylene glycol monomethyl ether, diethylene glycol monomethyl ether and monoethyl ether, and also aromatic alcohols, for instance benzyl alcohol or phenoxyethanol, and mixtures thereof.

According to a fourth aspect of this embodiment, the cosmetic composition A comprises one or more compounds chosen from agents that are capable of removing from the skin compounds that may be an obstacle to the action of one or more compounds of the cosmetic composition B.

In particular, the cosmetic composition A comprises one or more compounds that are capable of freeing the skin of salts that may be present, such as zinc.

According to a fifth aspect of this embodiment, the cosmetic composition A comprises one or more compounds that are capable of modifying the porosity of the skin.

In particular, the cosmetic composition A comprises one or more organic solvents and also products capable of breaking one or more hydrogen bonds, such as urea.

According to a sixth aspect of this embodiment, the cosmetic composition A comprises one or more compounds that are capable of making the skin reactive.

For example, the cosmetic composition A comprises one or more compounds chosen from oxidizing agents used under strict operating conditions so as to make the skin reactive.

In particular, use is made of an oxidizing agent referred to as a “mild oxidizing agent” that is not capable of fully oxidizing a third compound, unlike a “strong oxidizing agent”, which will fully degrade the third element. In the latter case, the oxidation product is degraded so much that it will no longer react.

According to a seventh aspect of this embodiment, the cosmetic composition A comprises one or more inhibitory compounds.

In particular, the cosmetic composition A comprises one or more zinc salts whose inhibitory action with respect to thiol functions prevents these functions from being reductive.

The compound(s) of the cosmetic composition A may also be chosen to withstand movements, elongations and rubbing.

Thus, the compound(s) of the cosmetic composition A make it possible especially to facilitate the penetration of one or more compounds of the cosmetic composition B into the skin, to increase their retention on the skin and to improve their functioning.

Preferably, the cosmetic composition A comprises one or more compounds that are capable of treating the skin by involving a chemical reaction.

More preferentially, the cosmetic composition A comprises one or more compounds chosen from cysteine salts, in particular cysteine hydrochloride, sulfite salts, in particular alkali metal or alkaline-earth metal sulfites such as sodium sulfite, alkoxysilanes of formula (I), in particular 3-aminopropyltriethoxysilane, manganese salts such as manganese chloride, thioglycolic acid, cationic polymers of formula (IX), or a mixture of these compounds.

The cosmetic composition B comprises one or more compounds selected from compounds that are capable of acting on the skin once it has been chemically or physically modified by the cosmetic composition A according to the invention, to give an antiperspirant effect.

For the purposes of the present invention, the expression “acting on the skin once it has been treated” means that the cosmetic composition B comprises one or more compounds that are capable of being active on contact with the skin once it has been treated with the cosmetic composition A.

According to a first embodiment, when the compound(s) of the cosmetic composition A are capable of being grafted onto the skin, then the compound(s) of the cosmetic composition B are selected from compounds that are capable of reacting with the compound(s) of the cosmetic composition A grafted onto the skin.

According to a second embodiment, when the compound(s) of the cosmetic composition A are capable of opening the disulfide bonds of the skin, thus forming reactive groups such as thiol groups, then the compound(s) of the cosmetic composition B are selected from compounds that are capable of reacting with the reactive groups thus formed, to impart an antiperspirant effect.

In accordance with this embodiment, the cosmetic composition B comprises one or more reducing agents such as cysteine.

Alternatively, in accordance with this embodiment, the cosmetic composition B comprises one or more compounds chosen from fluorinated alkoxysilanes.

According to a third embodiment, when the compound(s) of the cosmetic composition A are capable of modifying the ionic nature of the skin by forming one or more ionic groups, then the compound(s) of the cosmetic composition B are selected from compounds bearing one or more ionic charges of opposite sign to the ionic groups formed on the skin, so as to create an ionic interaction to impart an antiperspirant effect.

Thus, when the compound(s) of the cosmetic composition A are capable of modifying the ionic nature of the skin by forming one or more anionic groups, then the compound(s) of the cosmetic composition B are selected from compounds bearing one or more cationic charges capable of creating an ionic interaction with the negatively charged skin.

In other words, in this case, the compound(s) of the cosmetic composition B are selected so as to be able to react with the negatively charged skin by means of an anion/cation interaction.

Conversely, when the compound(s) of the cosmetic composition A are capable of modifying the ionic nature of the skin by forming cationic groups, then the compound(s) of the cosmetic composition B are selected from compounds bearing one or more anionic charges that are capable of creating an ionic interaction with the positively charged skin.

In other words, in this case, the compound(s) of the cosmetic composition B are selected so as to be able to react with the positively charged skin by means of a cation/anion interaction.

In accordance with this embodiment, the cosmetic composition B comprises one or more compounds that may be chosen from anionic polymers, metal salts, transition metal salts and/or cellulose esters.

Preferably, the anionic polymer(s) that may be used in the cosmetic composition B according to the invention comprise at least, as monomers, vinyl acetate, vinyl neodecanoate and crotonic acid.

The preferred polymer according to the invention is the terpolymer of vinyl acetate, of vinyl tert-butylbenzoate and of crotonic acid.

Such anionic polymers may be partially or totally neutralized with a basifying agent.

Preferably, the basifying agent is chosen from alkanolamines such as monoethanolamine, diethanolamine or triethanolamine, and derivatives thereof.

Among the alkali metal or alkaline-earth metal salts that may especially be used are halides, hydroxides, borates or nitrates, and more particularly alkali metal or alkaline-earth metal halides and borates, such as sodium chloride or sodium tetraborate.

Among the transition metal salts that may especially be used are transition metal halides, such as zinc chloride or manganese chloride.

Among the cellulose esters, it is particularly preferred to use hydroxypropylmethylcellulose phthalate.

According to another embodiment, when the compound(s) of the cosmetic composition A are capable of forming catalytic groups on the skin, then the compound(s) of the cosmetic composition B are selected from compounds that are capable of reacting in the presence of catalytic groups so as to impart an antiperspirant effect.

Thus, when the compound(s) of the cosmetic composition A are chosen from manganese salts, these compounds bind to the skin so as to give it catalytic activity that promotes oxidation and the compound(s) of the cosmetic composition B will then be selected from compounds that are capable of being oxidized, such as indoles, indole derivatives or thiol compounds so as to impart an antiperspirant effect.

According to another embodiment, when the compound(s) of the cosmetic composition A are compounds that are capable of modifying the porosity of the skin, then the compound(s) of the cosmetic composition B are selected from compounds that are capable of acting within the skin so as to impart an antiperspirant effect.

According to another embodiment, when the compound(s) of the cosmetic composition A are compounds that are capable of increasing the surface tension of the skin, then the compound(s) of the cosmetic composition B are selected from compounds that are capable of spreading on skin whose surface tension has been increased, so as to impart an antiperspirant effect.

According to another embodiment, when the compound(s) of the cosmetic composition A are solubilizers, then the compound(s) of the cosmetic composition B are selected from compounds that are capable of dissolving in the cosmetic composition A so as to impart an antiperspirant effect.

According to another embodiment, when the compound(s) of the cosmetic composition A are agents that are capable of modifying the hydrophobicity of the skin, then the cosmetic composition B comprises a volatile hydrophobic solvent and the compound(s) of the cosmetic composition B are chosen from compounds that are capable of entering the skin pores so as to impart an antiperspirant effect.

According to another embodiment, when the compound(s) of the cosmetic composition A are compounds that are capable of reducing the surface tension of the skin, then the cosmetic composition B comprises a liquid that has a high surface tension and the compound(s) of the cosmetic composition B are chosen from compounds that are capable of entering the skin pores so as to impart an antiperspirant effect. In particular, the compound(s) of the cosmetic composition B have a surface tension less than that of the compound(s) of the cosmetic composition B.

The compound(s) of the composition B may also be chosen from one or more antiperspirant compounds. Thus, the compound(s) of composition B may be chosen from aluminium salts, such as aluminium chloride and aluminium hydroxyhalides, or aluminium and zirconium complexes.

The compound(s) of composition B may also be chosen from one or more aluminium-based precursors capable of giving aluminium salts.

Thus, the compound(s) of composition B may be chosen from one or more aluminium salts in oligomerized or non-oligomerized hydrate form, in particular aluminium chlorohydrate.

The compound(s) of the cosmetic composition B may be chosen to withstand movements, elongations and rubbing.

For example, the compound(s) of the cosmetic composition B are chosen from compounds that are capable of withstanding abrasion, such as boron nitride.

Preferably, the cosmetic composition B comprises one or more compounds chosen from one or more fluorinated alkoxysilanes, one or more anionic polymers comprising at least, as monomers, vinyl acetate, vinyl neodecanoate and crotonic acid, cellulose esters, in particular hydroxypropylmethylcellulose phthalate, metal salts, in particular transition metal halides, reducing agents such as cysteine, indoles or derivatives thereof, aluminium chlorohydrate and sodium tetraborate.

Preferably, the cosmetic composition A comprises one or more compounds that are capable of modifying the ionic nature of the skin by creating cationic groups, and the cosmetic composition B comprises one or more compounds bearing one or more anionic charges capable of reacting with the cationic groups at the surface of the skin.

According to one embodiment, the cosmetic compositions A and B do not impart an antiperspirant effect.

For the purposes of the present invention, the expression “composition not having an antiperspirant effect” means a composition which, when applied to the surface of the skin, does not have the effect of significantly reducing or limiting the flow of sweat. In other words, such a composition, for the purposes of the present invention, has little influence on the flow of sweat.

For the purposes of the present invention, the expression “composition not having the effect of significantly reducing or limiting the flow of sweat” means a composition which, when applied to the skin, reduces the flow of sweat by a percentage, noted R, of less than 10%. The percentage reduction of the flow of sweat, noted R, is established in accordance with the in vivo Bioskin gravimetric test.

As a guide, the in vivo Bioskin gravimetric test gives the following scale:

| | | |
|---|---|---|
| percentage reduction | R < 10% | no efficacy |
| percentage reduction | 10 < R < 15% | low efficacy |
| percentage reduction | 15 < R < 25% | moderate efficacy |
| percentage reduction | 25 < R < 35% | good efficacy |
| percentage reduction | 35 < R < 50% | high efficacy |
| percentage reduction | R > 50% | very high efficacy |

Thus, according to the in vivo Bioskin gravimetric test, when the percentage reduction of the flow of sweat R is less than 10%, then the composition has no antiperspirant efficacy.

In this case, the combination of the cosmetic compositions A and B according to the invention can lead to an antiperspirant effect. Thus, the compound(s) of the cosmetic compositions A and B are selected so as to impart an antiperspirant effect in combination.

According to one aspect of this embodiment, the cosmetic composition A comprises one or more compounds chosen from cysteine salts, in particular cysteine hydrochloride, and the cosmetic composition B comprises one or more compounds chosen from metal salts, in particular transition metal salts such as zinc chloride.

According to one aspect of this embodiment, the cosmetic composition A comprises one or more compounds chosen from cysteine salts, in particular cysteine hydrochloride, and the cosmetic composition B comprises one or more compounds chosen from fluorinated alkoxysilanes.

According to another aspect of this embodiment, the cosmetic composition A comprises one or more compounds chosen from sulfites, in particular alkali metal or alkaline-earth metal sulfites such as sodium sulfites, and the cosmetic composition B comprises one or more compounds chosen from fluorinated alkoxysilanes.

According to another aspect of this embodiment, the cosmetic composition A comprises one or more compounds chosen from alkoxysilanes of formula (I) and the cosmetic composition B comprises one or more compounds chosen from magnesium chloride and anionic polymers comprising at least, as monomers, vinyl acetate, vinyl neodecanoate and crotonic acid.

According to another aspect of this embodiment, the cosmetic composition A comprises one or more compounds chosen from manganese salts, such as manganese chloride, and thioglycolic acid, and the cosmetic composition B comprises cysteine.

According to another aspect of this embodiment, the cosmetic composition A comprises one or more compounds chosen from manganese salts, such as manganese chloride, and thioglycolic acid, and the cosmetic composition B comprises dihydroxyindole.

Alternatively, according to another embodiment, the cosmetic compositions A and/or B may have an antiperspirant effect.

In this case, the combination of the cosmetic compositions A and B according to the invention can lead to an antiperspirant effect superior to that imparted by the cosmetic compositions A and B alone.

According to one aspect of this embodiment, the cosmetic composition A comprises one or more compounds chosen from cysteine salts, in particular cysteine hydrochloride, and the cosmetic composition B comprises aluminium chlorohydrate.

According to another aspect of this embodiment, the cosmetic composition A comprises one or more compounds chosen from the alkoxysilanes of formula (I), in particular 3-aminopropyltriethoxysilane, and the cosmetic composition B comprises hydroxypropylmethylcellulose phthalate.

According to another aspect of this embodiment, the cosmetic composition A comprises a tetramethylhexamethylenediamine polycondensate and the cosmetic composition B comprises sodium tetraborate.

According to another aspect of this embodiment, the cosmetic composition A comprises a tetramethylhexamethylenediamine polycondensate and the cosmetic composition B comprises aluminium chlorohydrate.

According to another aspect of this embodiment, the cosmetic composition A comprises one or more compounds chosen from cysteine salts, such as cysteine hydrochloride, and alkali metal or alkaline-earth metal sulfites such as sodium sulfite, and the cosmetic composition B comprises aluminium chlorohydrate.

The cosmetic composition(s) A and/or B may especially be in the form of a powder, a suspension, a dispersion, a solution, a gel, an emulsion, especially an oil-in-water (O/W) or water-in-oil (W/O) or multiple (W/O/W or polyol/O/W or O/W/O) emulsion, or in the form of a cream, a mousse, a stick, a dispersion of vesicles, especially of ionic or nonionic lipids, a two-phase or multiphase lotion, a spray, a powder, a paste or an adhesive or non-adhesive film.

When the cosmetic composition(s) A and/or B are conditioned in an aerosol device, it comprises one or more propellants, which may be chosen from volatile hydrocarbons such as n-butane, propane, isobutane, pentane and halogenated hydrocarbons, and mixtures thereof.

Carbon dioxide, nitrous oxide, dimethyl ether (DME), nitrogen or compressed air may also be used as propellant. Mixtures of propellants may also be used. Dimethyl ether is preferably used.

Advantageously, the propellant may be present in a concentration of between 5% and 90% by weight and more particularly in a concentration of between 10% and 60% by weight relative to the total weight of the composition in the aerosol device.

A person skilled in the art can select the appropriate galenical form, and also the method for preparing it, on the basis of his general knowledge, taking into account firstly the nature of the constituents used, especially their solubility in the support, and secondly the intended use of the composition.

Preferably, the cosmetic compositions A and/or B are in the form of a solution, an oil-in-water (O/W) or water-in-oil (W/O) emulsion or in the form of creams or gels.

Alternatively, the cosmetic compositions A and/or B are conditioned in an aerosol device that comprises one or more propellants and especially nitrogen and/or dimethyl ether (DME).

The cosmetic compositions A and B comprise a cosmetically acceptable medium.

The medium that is suitable for the cosmetic compositions A and B of the invention is a cosmetically acceptable medium generally comprising water or a mixture of water and at least one organic solvent. Examples of organic solvents that may be mentioned include C₁-C₄ lower alkanols, such as ethanol and isopropanol; polyols and polyol ethers such as 2-butoxyethanol, propylene glycol, glycerol, propylene glycol monomethyl ether, diethylene glycol monomethyl ether and monoethyl ether, and also aromatic alcohols, for instance benzyl alcohol or phenoxyethanol, and mixtures thereof.

The solvents are preferably present in proportions of between 1% and 40% by weight approximately and even more preferentially between 5% and 30% by weight approximately relative to the total weight of the cosmetic composition.

The cosmetic compositions A and/or B may also contain one or more cosmetic adjuvants chosen, for example, from catalysts, deodorant active agents, fatty substances, softeners, antifoams, moisturizers, UV-screening agents, mineral colloids, peptizers, colorants such as ninhydrin, solubilizers, fragrances, nonionic or amphoteric surfactants, proteins, vitamins, propellants, oxyethylenated or non-oxyethylenated waxes, paraffins, C10-C30 fatty acids such as stearic acid or lauric acid, and C10-C30 fatty amides such as lauric acid diethanolamide.

The above additives are generally present in an amount for each of them of between 0.01% and 20% by weight relative to the weight of the composition.

Moreover, the present invention also relates to a cosmetic process for treating human perspiration, which consists in applying to the skin simultaneously, separately or sequentially over time a cosmetic composition A comprising one or more compounds that are capable of treating the skin, and a cosmetic composition B different from the cosmetic composition A, comprising one or more compounds that are capable of acting on the skin once it has been treated, such that their combination is capable of imparting an antiperspirant effect.

The cosmetic compositions A and B may be applied several times to the skin.

When the cosmetic compositions A and/or B are applied to the skin sequentially over time, the time interval between the application of the cosmetic composition A and the application of the cosmetic composition B may be between 1 second and 1 hour, preferably between 10 seconds and 20 minutes and even more preferentially between 2 minutes and 6 minutes.

Moreover, the cosmetic composition A may be applied in the morning, optionally several times, and the cosmetic composition B may be applied in the evening.

According to one embodiment, the skin is rinsed between the application of the cosmetic composition A and the application of the cosmetic composition B.

Preferably, the skin is not rinsed between the application of the cosmetic composition A and the application of the cosmetic composition B.

It should be noted that when the cosmetic compositions A and B are applied separately, the said cosmetic compositions may be in identical galenical forms or different galenical forms.

According to one embodiment, the application of the multi-component antiperspirant agent may be performed on the skin before or after applying a pretreatment or post-treatment composition.

Another subject of the present invention concerns a multi-compartment device or kit comprising a first compartment including a cosmetic composition A and a second compartment including a cosmetic composition B different from the cosmetic composition A, the said cosmetic composition A comprising one or more compounds that are capable of treating the skin, and a cosmetic composition B different from the cosmetic composition A, comprising one or more compounds that are capable of acting on the skin once it has been treated, such that their combination is capable of imparting an antiperspirant effect.

The multi-compartment device or kit may be a pump bottle, a tube or a stick comprising two applicators.

It should be noted that when the cosmetic compositions A and B are applied separately, the said cosmetic compositions may be in identical galenical forms or different galenical forms.

The invention also relates to the use of a multi-component agent as defined above for treating human perspiration.

The examples that follow serve to illustrate the present invention.

I. In the Case where the Cosmetic Compositions A and B have No Antiperspirant Activity

### EXAMPLE 1

The two compositions below are prepared (unless otherwise mentioned, the amounts are expressed as weight percentages):

| | |
|---|---|
| Composition A | |
| Cysteine hydrochloride | 7% |
| Aqueous ammonia | qs pH 9 |
| water | qs 100% |
| Composition B | |
| Zinc chloride | 7% |
| Citric acid | qs pH 4 |
| Ethanol | 20 g |
| water | qs 100% |

0.7 g of the cosmetic composition A is applied to a preshaved armpit and is left to stand for 1 minute. 0.4 g of the cosmetic composition B is then applied to the same area. The armpit is massaged, and rinsed after 1 minute.

### EXAMPLE 2

The two compositions below are prepared (unless otherwise mentioned, the amounts are expressed as weight percentages):

| | |
|---|---|
| Composition A | |
| Cysteine hydrochloride | 7% |
| Aqueous ammonia | qs pH 9 |
| water | qs 100% |
| Composition B | |
| Fluoroalkanesilane | 1.2% |
| triethoxy(3,3′,4,4′,5,5′,6,6′,7,7′,8,8′,8″- | |
| tridecafluorooctyl)silane | |
| Ethanol | qs 100% |

1 g of composition A is applied to a delimited area of 4×4 cm of skin of the forearm of a first model. The composition is left to stand for 1 minute and is then rinsed off. Next, 1 g of composition B of Example 2 is applied to the same area. The composition is dried with a hairdryer for 3 minutes.

### EXAMPLE 3

The following compositions are prepared (unless otherwise mentioned, the amounts are expressed as weight percentages):

| | |
|---|---|
| Composition A | |
| Sodium sulfite | 5% |
| Sodium hydroxide | qs pH 6.9 |
| water | qs 100% |
| Composition B | |
| Fluoroalkanesilane | 1.2% |
| triethoxy(3,3′,4,4′,5,5′,6,6′,7,7′,8,8′,8″- | |
| tridecafluorooctyl)silane | |
| Ethanol | qs 100% |

1 g of composition A is applied to a delimited area of 4×4 cm of forearm skin of a first model. The composition is left to stand for 1 minute and is then dabbed to remove the excess. 0.4 g of composition B is then applied to the same area. The forearm is massaged and dried with a hairdryer for 5 minutes.

### EXAMPLE 4

The following compositions are prepared (unless otherwise mentioned, the amounts are expressed as weight percentages):

| | |
|---|---|
| Composition A | |
| 3-Aminopropylethoxysilane | 10% |
| (from Dow Corning) | |
| Sulfuric acid | qs pH 9.7 |
| water | qs 100% |
| Composition B | |
| Magnesium chloride | 1.2% |
| Anionic resin | 10% |
| (Vinyl acetate/crotonic acid/vinyl | |
| neodecanoate terpolymer) sold by | |
| National Starch | |
| Ethanol | 20% |
| Monoethanolamine | qs 80% neutralization |
| | of the polymer |
| Water | qs 100% |

1 g of the cosmetic composition A is applied to the forearm of a first model (delimited area of 4 cm×4 cm). The composition is left to stand for 1 minute and is then dabbed to remove the excess. 0.4 g of the cosmetic composition B is then applied to the same area. The forearm is massaged and dried with a hairdryer for 2 minutes.

### EXAMPLE 5

The following compositions are prepared in the absence of air, and are maintained under nitrogen. Unless otherwise indicated, the amounts are expressed as weight percentages:

| Composition A | |
|---|---|
| Manganese chloride | 10% |
| Thioglycolic acid | 3% |
| Aqueous ammonia | qs pH 8.5 |
| Water | qs 100% |
| Composition B | |
| Cysteine | 4% |
| Aqueous ammonia | qs pH 9 |
| Ethanol | 100% |

1 g of composition A is applied to the forearm of a first model (delimited area of 4 cm×4 cm). The composition is left to stand for 3 minutes. The excess is dabbed off and the forearm is then rinsed. A spray formed from a 10-volumes aqueous hydrogen peroxide mist is then applied to the treated area, and is left to dry.

0.4 g of composition B is then applied to the same area several times in the morning, 30 minutes before showering. The composition is rinsed off each time under the shower.

### EXAMPLE 6

The following compositions are prepared in the absence of air, and are maintained under nitrogen. Unless otherwise indicated, the amounts are expressed as weight percentages:

| Composition A | |
|---|---|
| Manganese chloride | 10% |
| Thioglycolic acid | 3% |
| Aqueous ammonia | qs pH 8.5 |
| Water | qs 100% |
| Composition B | |
| Dihydroxyindole | 1% |
| Ethanol | 20% |
| Water | 100% |

The same tests as in Example 5 are performed.

II. In the Case where the Cosmetic Composition A and/or B has Antiperspirant Activity

### EXAMPLE 1

The following compositions are prepared (unless otherwise mentioned, the amounts are expressed as weight percentages):

| | |
|---|---|
| Composition A | |
| Sodium sulfite | 5% |
| Sodium hydroxide | qs pH 6.9 |
| water | qs 100% |
| Composition B | |
| Aluminium chlorohydrate | 10% |
| Ethanol | qs 100% |

1 g of the cosmetic composition A is applied to the forearm of a first model (delimited area of 4 cm×4 cm). The composition is left to stand for 1 minute and is then dabbed to remove the excess. Next, 1 g of the cosmetic composition B is applied to the same area. The forearm is massaged and dried with a hairdryer for 5 minutes.

### EXAMPLE 2

The following compositions are prepared (unless otherwise mentioned, the amounts are expressed as weight percentages):

| | |
|---|---|
| Composition A | |
| 3-Aminopropylethoxysilane | 10% |
| (from Dow Corning) | |
| Sulfuric acid | qs pH 9.7 |
| water | qs 100% |
| Composition B | |
| Hydroxypropylmethylcellulose | 3% |
| phthalate | |
| Water | 20% |
| Dimethyl ether | 35% |
| Ethanol | qs 100% |

1 g of the cosmetic composition A is applied to the forearm of a first model (delimited area of 4 cm×4 cm). The composition is left to stand for 1 minute and is then dabbed to remove the excess. 0.4 g of the cosmetic composition B is then applied to the same area. The forearm is massaged and dried with a hairdryer for 2 minutes.

### EXAMPLE 3

The following compositions are prepared (unless otherwise mentioned, the amounts are expressed as weight percentages):

| Composition A | |
|---|---|
| Tetramethylhexamethyldiamine/1,3- | 1.5% |
| dichloropropylene polycondensate | |
| as an aqueous 60% solution | |
| Water | qs 100% |
| Composition B | |
| Sodium tetraborate | 4% |
| sold by Sigma-Aldrich | |
| Water | 20% |

1 g of composition A is applied to the forearm of a first model (delimited area of 4 cm×4 cm). The composition is left to stand for 3 minutes. The excess is dabbed off and the forearm is then rinsed. 2 g of composition B are then applied to the same area in the morning, 30 minutes before showering. The composition is rinsed off under the shower.

### EXAMPLE 4

The following compositions are prepared (unless otherwise mentioned, the amounts are expressed as weight percentages):

| Composition A | |
|---|---|
| Tetramethylhexamethyldiamine/1,3- | 1.5% |
| dichloropropylene polycondensate | |
| as an aqueous 60% solution | |
| Water | qs 100% |
| Composition B | |
| Aluminium chlorohydrate | 5% |
| Ethanol | 20% |

1 g of composition A is applied to the forearm of a first model (delimited area of 4 cm×4 cm). The composition is left to stand for 3 minutes. The excess is dabbed off and the forearm is then rinsed under the shower.

### EXAMPLE 5

| | |
|---|---|
| Composition A | |
| Sodium sulfite | 7% |
| Cysteine hydrochloride | 7% |
| Sodium hydroxide | qs pH 8.5 |
| Water | qs 100% |
| Composition B | |
| Aluminium chlorohydrate | 10% |
| Ethanol | qs 100% |

1 g of composition A is applied to the forearm of a first model (delimited area of 4 cm×4 cm). The composition is left to stand for 3 minutes. The excess is dabbed off and the forearm is then rinsed under the shower.

1. A multi-component antiperspirant agent, comprising:
a first component cosmetic composition A, comprising one or more compounds that are capable of treating the skin; and
a second component cosmetic composition B, different from the composition A, comprising one or more compounds that are capable of acting on the skin once it has been treated;
which are intended to be combined by application to the skin separately or sequentially over time, as an antiperspirant.

2. The multi-component antiperspirant agent according to claim 1, wherein the cosmetic composition A comprises one or more compounds which treat the skin by a chemical reaction.

3. The multi-component antiperspirant agent according to claim 1, wherein the cosmetic composition A comprises one or more compounds selected from the group consisting of compounds which graft to the skin, compounds which open one or more bonds of the skin, compounds which modify the ionic nature of the skin, compounds which remove all or some of the molecules present in the skin, compounds which generate catalytic groups on the skin and a mixture of these compounds.

4. The multi-component antiperspirant agent according to claim 1, wherein the cosmetic composition A comprises one or more compounds selected from the group consisting of cysteine salts,
sulfite salts,
manganese salts,
thioglycolic acid, and alkoxysilanes of formula (I) comprising a primary amine: 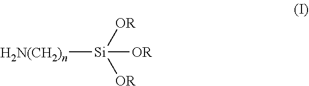
wherein R is independently C1-C6 alkyl and n is an integer from 1 to 6.

5. The multi-component antiperspirant agent according to claim 1, wherein the cosmetic composition A comprises one or more compounds which treat the skin without chemical reaction with the skin.

6. The multi-component antiperspirant agent according to claim 5, wherein the cosmetic composition A comprises one or more compounds selected from the group of compounds consisting of compounds which increase a retention on the skin of one or more compounds of the cosmetic composition B,
compounds which remove agents which harm an action of the compound(s) of the cosmetic composition B,
compounds which improve at least one of a distribution on the skin and a penetration in the skin of one or more compounds of the cosmetic composition B.

7. The multi-component antiperspirant agent according to claim 1, wherein the one or more compound of the cosmetic composition A opens disulfide bonds of the skin, thus forming reactive groups on the surface of the skin, and the one or more compound of the cosmetic composition B is selected from the group of compounds which react with the formed reactive groups.

8. The multi-component antiperspirant agent according to claim 3, wherein when the compound(s) of the cosmetic composition A form one or more catalytic groups on the skin, then the compound(s) of the cosmetic composition B are selected from the group consisting of compounds which react in the presence of the catalytic groups to impart an antiperspirant effect.

9. The multi-component antiperspirant agent according claim 3, wherein when the compound(s) of the cosmetic composition A modify the ionic nature of the skin by forming cationic groups, then the compound(s) of the cosmetic composition B are selected from the group consisting of compounds bearing one or more anionic charges and ionically interact with the cationic groups of the skin.

10. The multi-component antiperspirant agent according to claim 1, wherein the cosmetic composition B comprises one or more compounds selected from the group consisting of fluorinated alkoxysilanes, anionic polymers comprising at least, as monomers, vinyl acetate, vinyl neodecanoate and crotonic acid, cellulose esters, metal salts, reducing agents, aluminium chlorohydrate and sodium tetraborate.

11. The multi-component antiperspirant agent according to claim 1, wherein each of the cosmetic compositions A and B has or does not have an antiperspirant effect.

12. A process for treating human perspiration, comprising:
applying to the skin simultaneously, separately or sequentially over time a cosmetic composition A and a cosmetic composition B different from the cosmetic composition A,
wherein
the cosmetic composition A comprises one or more compounds which the skin, and
the cosmetic composition B comprises one or more compounds that act on the treated, and
the combination of compositions A and B imparts an antiperspirant effect to the skin.

13. The process according to claim 12, wherein there is a time interval between the application of the cosmetic composition A and the application of the cosmetic composition B.

14. A method for treating human perspiration, comprising application of the multi-component antiperspirant agent according to claim 1.

15. A multi-compartment device or kit comprising:
a first compartment including a cosmetic composition A, comprising one or more compounds which treats the skin and
a second compartment including a cosmetic composition B different from the cosmetic composition A, comprising one or more compounds that acts on the treated skin
wherein composition A and composition B are combined by application to the skin simultaneously, separately or sequentially over time, and the combination imparts an antiperspirant effect on the skin.

16. The multi-component antiperspirant agent according to claim 10, wherein the cosmetic composition B comprises at least one reducing agent selected from the group consisting of cysteine, an indole and a derivatives of cysteine or indole.
